(19) United States
(12) Patent Application Publication
(10) Pub. No.: US 2007/0292836 A1
(43) Pub. Date: Dec. 20, 2007

(54) Methods for sorting undifferentiated cells and uses thereof

(75) Inventors: Tomoyuki Tokunaga, Tsukuba-shi (JP); Tadashi Furusawa, Tsukuba-shi (JP); Chris Honda, Tsukuba-shi (JP); Katsuhiro Ohkoshi, Tsukuba-shi (JP)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: National Institute of Agrobiological Sciences, Tsukuba-shi (JP)

(21) Appl. No.: 11/804,050

(22) Filed: May 16, 2007

Related U.S. Application Data
(63) Continuation of application No. 10/719,702, filed on Nov. 21, 2003, now abandoned.

(30) Foreign Application Priority Data
Mar. 28, 2003 (JP) 2003-092465

(51) Int. Cl. A01K 67/00; A01N 1/00; C12N 15/00; C12N 5/06; G01N 33/567

(52) U.S. Cl. 435/1.1; 435/354; 435/7.21; 800/21; 800/8

(57) ABSTRACT

A method of sorting undifferentiated cells is provided. Subpopulations of cells contained in a single line of ES cells can be sorted using cell-surface markers. The sorted undifferentiated cells have ability to produce chimera with high contribution and germ line transmission efficiently. These undifferentiated cells can be used as a valuable vehicle for the production of transgenic animals and also knockout animals.

### RELATED APPLICATIONS

This application is a continuation of U.S. application Ser. No. 10/719,702, filed Nov. 21, 2003, which claims priority under 35 U.S.C. §119 or 365 to Japan Application No. 2003-092465, filed Mar. 28, 2003. The entire teachings of the above applications are incorporated herein by reference.

### FIELD OF THE INVENTION

This invention relates to a method for sorting undifferentiated cells, wherein said method comprises contacting the cells with antibodies to cell-surface antigens and isolating only cells bound to the antibodies. The isolated cells efficiently produce chimeric and cloned individuals and such.

### BACKGROUND OF THE INVENTION

Undifferentiated cells, such as embryonic stem (ES) cells or embryonic germ (EG) cells, and transgenic undifferentiated cells are cultured under appropriate culture conditions, and then separated into single cells by enzymatic treatment. To produce chimeric embryos, about five to ten separated cells are collected at random and injected into normal blastocysts or aggregated with the cleavage stage embryos including tetraploid embryos. After transferred these chimeric embryos into the oviducts or uteri of surrogate mothers, they develop to term and chimeric offspring are delivered. The genotype of the original undifferentiated cells can be inherited to the plus when these chimeric animals are mated, if these cells have contributed to their germ cells, which differentiate into eggs and sperm. Therefore, undifferentiated cells can be used as a vehicle for in the production of transgenic animals. This is indispensable for the production of knockout animals in which the function of a specific gene has been disrupted.

When used as donor cells for nuclear transplantation, undifferentiated cells or genetically engineered undifferentiated cells can directly develop into individual animals. To this end, an unfertilized oocyte is enucleated and then fused with an ES cell using a cell fusion technique such as an electric pulse. Alternatively, the nucleus of an ES cell can be directly inserted into an unfertilized enucleated oocyte, using an insertion device with a piezo-drive. With appropriate developmental stimulation, an oocyte with a transplanted nucleus will begin to develop. The oocyte is then transferred into the oviduct of a surrogate mother, and an individual cloned from the donor ES cell can be obtained. The cloned individual's genetic information is totally derived from the undifferentiated donor cell. Thus if the donor cell is transgenic, the cloned individual itself will be transgenic, and the need to wait for the breeding and production of transgenic offspring is eliminated.

Putting technical factors aside, successful chimera formation is thought to depend on the characteristics of the ES cell line used. Many cell lines can not produce chimeras. Even in cell lines that do produce chimeras relatively readily, chimera production is generally in the range of several to less than twenty per cent. In addition, to produce knockout animals, the ES cells must contribute to the germ line of the chimeric animal. This is problematic due to the extremely low efficiency of this process, and thus the production of knockout animals and the like becomes enormously expensive. Similarly, the production of individuals cloned from ES cells is also inefficient, being in the range of several percent. Normality of the cloned individuals is also low.

### SUMMARY OF THE INVENTION

An objective of this invention is to improve the efficiency of production of chimeric individuals by using undifferentiated cells. Another objective is to improve the degree of contribution of undifferentiated cells in chimeras, and to make it easier to obtain germline chimeras. Still another objective of this invention is to improve the efficiency with which normal cloned individuals are produced when undifferentiated cells are used as donor cells for nuclear transplantation.

ES cells are derived from an inner cell mass of blastocysts and maintain a high degree of pluripotency. ES cells are not a uniform population of cells, but rather a mixture of cells with varying potential for differentiation and chimera formation. Based on this concept, the present inventors premised that fractionation of cell subpopulations would enable comparison between completely undifferentiated cells, and cells which have just begun to differentiate into specific cell lines. The inventors also concluded that fractionation would be of use in elucidating the pluripotency maintenance mechanism and differentiation induction factors in ES cells.

Based on this idea, the present inventors derived ES cells from ROSA26×CBA mice and then stained these cells with various cell-surface marker antibodies. Subsequent flow cytometry analysis revealed large variances in the expression of PECAM-1 and SSEA-1. Thus the inventors double stained ES cells with PECAM-1 and SSEA-1, and then cells were sorted into three subpopulations exhibiting PECAM-1⁻ SSEA-1⁻, PECAM-1⁺SSEA-1⁻, and PECAM-1⁺SSEA-1⁺ using FACS. RNA from each subpopulation was purified, and gene expression profiles were compared using quantitative RT-PCR. In addition, one cell from each of these subpopulations was injected into an eight-cell embryo, and localization of β-gal-positive cells in the chimeric embryo was investigated. The results revealed that almost all SSEA-1 positive cells were also PECAM-1 positive. Comparison of gene expression showed that PECAM-1 negative cells showed increased expression of differentiation markers in the primitive endoderm, ectoderm, and mesoderm. In contrast, gene expression of Oct3/4 was reduced. Analysis of localization of ES cell-derived cells in the blastocyst indicated a high frequency of PECAM-1 positive cells in the epiblast. Localization of PECAM-1 negative cells in the primitive endoderm or trophectoderm was also observed with high frequency. Moreover, in 6.0 to 7.0 d. p. c. embryos, only PECAM-1⁺SSEA-1⁺ cells differentiated into the epiblasts at a high frequency. These facts suggest that the expression of PECAM-1 and SSEA-1 is closely associated with the pluripotency of ES cells.

Thus, the present inventors found that within a single ES cell line there are subpopulations of cells with different characteristics, and that these subpopulations can be fractionated by cell-surface markers. The inventors also found that differences in the efficiency of chimera formation is not due to differences in characteristics of the ES cell line used, but rather is a reflection of differences in the composition of the stem cell subpopulations. The present invention is based on these findings. This invention enables the selection of appropriate cell-surface markers, the sorting of ES cells using these markers, and the isolation of cell subpopulations that produce chimeras efficiently.

This invention relates to a method of sorting out undifferentiated cells having characteristics such as the potential for efficient chimera formation and the ability to form normal individuals following nuclear transplantation. More specifically, this invention relates to the following:

(1) a method of sorting undifferentiated cells, wherein said method comprises contacting undifferentiated cells with an antibody to a cell-surface antigen, and sorting the undifferentiated cells according to the presence or absence of the binding to the antibody,

(2) the method of (1), wherein the cell-surface antigen is selected from the group consisting of PECAM-1, SSEA-1, SSEA-3, and SSEA-4,

(3) the method of (1), wherein the undifferentiated cells are embryonic stem (ES) cells derived from mammals or embryonic germ (EG) cells derived from mammals,

(4) the method of (1), wherein the undifferentiated cells are transgenic,

(5) the method of (2), wherein undifferentiated cells that bind to antibody to PECAM-1 is sorted out,

(6) the method of (5), wherein undifferentiated cells that bind to both an antibody to PECAM-1 and an antibody to SSEA-1 are sorted out,

(7) the method of (5), wherein undifferentiated cells that bind to an antibody to PECAM-1, an antibody to SSEA-3, and an antibody to SSEA-4 are sorted out,

(8) an undifferentiated cell obtained by the method of any one of (5), (6), and (7),

(9) an isolated undifferentiated cell binding to an antibody to PECAM-1,

(10) an isolated undifferentiated cell binding to an antibody to PECAM-1 and an antibody to SSEA-1,

(11) an isolated undifferentiated cell binding to an antibody to PECAM-1, an antibody to SSEA-3, and an antibody to SSEA-4,

(12) a host embryo comprising the undifferentiated cell of (8),

(13) the embryo of (12), wherein the embryo is constructed by injection of the undifferentiated cell of (8) into a fertilized embryo, or aggregation of that undifferentiated cell with a fertilized embryo,

(14) the embryo of (12), wherein said embryo is constructed by injection or fusion of the undifferentiated cell of (8) to an enucleated unfertilized oocyte,

(15) a differentiated tissue complex derived from the undifferentiated cell of (8),

(16) the differentiated tissue complex of (15), wherein said complex is constructed by inducing differentiation by in vitro culture or in vivo transplantation of the undifferentiated cell of (8),

(17) a surrogate mother comprising the embryo of (12), and

(18) a fetus, baby, or descendants obtained from the surrogate mother of (17).

### BRIEF DESCRIPTION OF THE DRAWINGS

FIG. 1. A: The double staining patterns of ES cells (ES), EG cells TM1 (EG), and embryonal carcinoma cells, F9 (EC) by PECAM-1 and SSEA-1. B: ES cells were separated into three subpopulations, cultured for four days, and then collected. B shows the occurrence of cells from other subpopulations in these cultures.

FIG. 2 shows the expression of genes involved in the regulation and reflection of differentiation with respect to the populations PECAM-1⁻SSEA-1⁻, PECAM-1⁺SSEA-1⁻, and PECAM-1⁺SSEA-1⁺.

FIG. 3. A: Time-course of the expression of PECAM-1 in ES cells. B: Time-courses of the gene expression of Oct3/4, Pecam-1, Rex1, and Hand1.

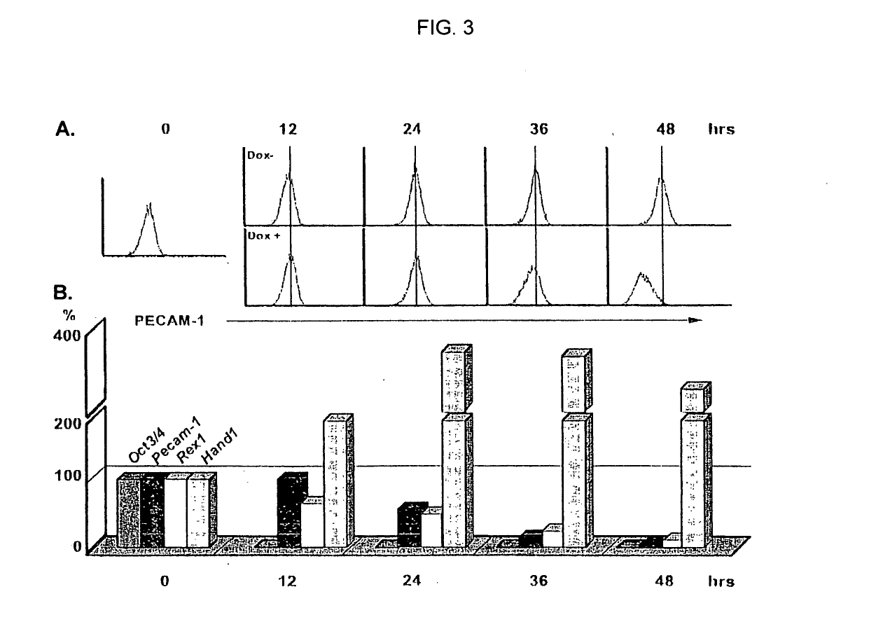

FIG. 4 presents photographs of X-gal-stained embryos 36 hours after injection of ES cells. The lower photograph is a magnification of the top part of the upper photograph. The arrow shows derivatives from a PECAM-1⁺SSEA-1⁺ cell.

FIG. 5 schematically shows a mouse embryo at 6.0 and 7.0 d. p. c.

FIG. 6 shows photographs of the X-gal-stained embryos recovered four days after transplantation. The lower photograph shows a cross-section along a line in v. iv and v show PECAM-1⁺SSEA-1⁺ cell-injected host embryos.

### DETAILED DESCRIPTION OF THE INVENTION

This invention relates to a method of selecting and sorting for undifferentiated cells that have characteristics such as the potential for efficient chimera formation and the ability to form normal individuals following nuclear transplantation. The undifferentiated cells of this invention include ES cells and EG cells. These cells can be from any animal, without restriction. The cells may also be genetically engineered cells used in the production of transgenic animals (for example, undifferentiated cells where genes have been integrated into the genome by homologous recombination). Undifferentiated cells are selected using cell-surface antigens as indicators. Generally, cells are contacted with antibodies to their cell-surface antigens, and then selected by the presence or absence of the binding to the antibodies. Any cell-surface antigen can be used, so long as it can select undifferentiated cells with high regeneration abilities, such as the ability to form chimeras efficiently or generate normal individuals following nuclear transplantation. However, cell-surface antigens selected from the group consisting of PECAM-1, SSEA-1, SSEA-3, and SSEA-4 are preferred. For example, in the case of a mouse, PECAM-1 and SSEA-1 are preferably used as cell-surface markers that are contacted with specific antibodies. In some animal species, such as humans, SSEA-1 cannot be detected in undifferentiated cells. In these cases, SSEA-3 and SSEA-4 are used in preference to SSEA-1. There are no particular limitations as to the methods used to sort out undifferentiated cells after specific antibody binding. In most cases, fluorescence excitation cell sorters are used. A simpler cell-sorting apparatus, such as one that uses immunomagnetic beads, can also be used.

For example, fluorescence-excited cells can be sorted as follows: Cells under cultivation are treated with Accutase enzyme solution (Innovative Cell Technologies, La Jolla, Calif., USA) diluted four times with PBS. Following separation into single cells, the cells are collected by centrifugation, resuspended in PBS with 0.2% bovine serum albumin, and then stained with antibodies. For example, in the double staining with PECAM-1 and SSEA-1, R-phycoerythrin-labeled anti-mouse PECAM-1 antibody and anti-SSEA-1 antibody are added to the cell suspension, and then cooled on ice for 30 minutes. The cells are then washed using PBS with 0.2% bovine serum albumin, and resuspended. Fluorescein isothiocyanate (FITC)-labeled anti-mouse IgM antibody is added to the cell suspension, which is then cooled on ice for 30 minutes. The cells are washed using PBS with 0.2% bovine serum albumin, resuspended, and then fluorescence-excited cells are sorted. Alternatively, cell sorting can be carried out using immunomagnetic beads, by combining cells with a primary antibody (labeled anti-mouse PECAM-1 antibody or anti-SSEA-1 antibody) and then binding anti-mouse IgG antibody-coated magnetic beads, or anti-mouse IgM antibody-coated magnetic beads to the cells.

Sorted undifferentiated cells can be used for the construction of chimeric embryos. There are two general methods for chimeric embryo construction using sorted undifferentiated cells, depending on the developmental stage of the host embryo (refer to “Operational Manual for Mouse Embryos”, translated by Kazuya Yamanouchi et al., Kindai Shuppan (1994); “Gene Targeting” by Shin-ichi Aizawa, Yodo-sha (1995); “Methods of Genetic Engineering in Embryos and Adults of Animals” edited by Yasuhito Kondo, Springer-Verlag Tokyo (1997); “Recent Techniques of Gene Targeting” edited by Takeshi Yagi, Yodo-sha (2000)). When the host is in the blastocyst stage, a micro-manipulator can be used to maneuver a glass injection pipette, injecting one to ten ES cells into the blastocele (an inner space formed in the blastocyst) (the so-called injection method). When the host is in a cleavage stage (in most cases the eight-cell stage), the embryo is treated with acidic Tyrode solution and the zona pellucida is removed. The embryo thus treated and ES cells are then aggregated (the so-called aggregation method). In some cases, the zona pellucida is not removed and the ES cells are injected into the perivitelline space (the space between the zona pellucida and the embryo).

The undifferentiated cells, once sorted, can also be used to construct nuclear-transplanted embryos. Techniques for the construction of such embryos using undifferentiated cells, are described below (refer to “Newest Techniques of Gene Targeting” edited by Takeshi Yagi, Yodo-sha (2000); “Protocols in Stem Cell and Clone Research” edited by Norio Nakatsuji, Yodo-sha (2001)): First, chromosomes in the metaphase of their second meiotic division are removed from the mature oocyte of a mouse by suction with a glass pipette, maneuvered with a micromanipulator. The nucleus of the ES cell is sucked into an injection pipette mounted on a piezo-drive. The nucleus is then injected into the cytoplasm of the enucleated oocyte. In an alternative method, an ES cell, together with inactivated HVJ virus particles, is injected into the perivitelline space of an enucleated oocyte. The nucleus of the ES cell is then transplanted into the cytoplasm of the oocyte by cell fusion. Instead of HVJ, an electric pulse can be used to induce fusion of the ES cell and the oocyte. The nuclear-transplanted embryos thus prepared can be transferred into surrogate mothers and hence develop into fetuses and offspring. This invention includes these surrogate mothers into which nuclear-transplanted embryos have been transferred, as well as fetuses and offspring obtained from such surrogate mothers, and any descendants obtained from the fetuses and offspring.

A differentiated tissue complex is a cellular structure containing various differentiated tissues derived from undifferentiated cells. It can be constructed by inducing differentiation of the sorted undifferentiated cells, using in vitro culture or in vivo transplantation (refer to “Protocols in Stem Cell and Clone Research” edited by Norio Nakatsuji, Yodo-sha (2001)). For example, ES cells injected into an immunodeficient mouse hypodermically or under the kidney membrane, can cause formation of teratomas containing differentiated tissues such as muscle, heart, and bone tissues.

In addition to in vivo transplantation, a differentiated tissue complex can also be constructed by in vitro culture, where differentiation is induced using culture dishes without transplantation into living organisms. For example, cell differentiation suppression factors, such as feeder cells and LIF, can be removed from the usual ES cell culture system. ES cells can be dissociated into single-cell suspension, then cultured to form a differentiated tissue complex called an embryoid body. An embryoid body has a relatively simple structure and includes tissues differentiated into three germ layers. Continued culture of this embryoid body can be used to obtain differentiated cells such as neural cells and blood cells. To achieve this end, the embryoid body is cultured on a matrix such as laminin or fibronectin, and using a medium containing differentiation-inducing substances such as retinoic acid and activin. Differentiation can also be induced and desired cells directly obtained from ES cells by combining special feeder cells and growth factors, etc. Furthermore, differentiated tissue complexes, similar to the teratomas obtained by in vivo transplantation of the ES cells, can be obtained by culturing the ES cells on, for example, a multifunctional three-dimensional culture matrix using collagen gel or gauze.

Chimeric embryos, nuclear-transplanted embryos, and differentiated tissue complexes can be constructed using EG cells, in the same way as for ES cells.

The present invention enables the sorting of undifferentiated cells using cell-surface markers as indicators, thereby isolating cell populations that can produce chimeras efficiently. These undifferentiated cells can be used as a vehicle for the production of transgenic animals. When undifferentiated cells sorted by the methods of this invention are used in this way, highly efficient and reduced cost production of transgenic animals is possible.

### EXAMPLES

The present invention is illustrated in detail below with reference to Examples, but is not to be construed as being limited thereto.

### Example 1

### Subpopulations with Differing Degrees of PECAM-1 and SSEA-1 Expression Exist in ES Cells

ES cells derived from the blastocysts of ROSA26×CBA F₁ mice, and EG cells (TM1), were each cultured on a feeder cell layer (STO) in ES medium with 20% KSR (GIBCO BRL). Embryonal carcinoma (EC) cells (F9) were cultured on gelatin-coated dishes containing high-glucose DME medium with 2-ME and NEAA. The cells obtained were dispersed by treatment with Accutase (Innovative Cell Technologies) and then stained with phycoerythrin-labeled anti-PECAM-1 antibody (40 ng/10⁶ cells, Pharmingen) and anti-SSEA-1 antibody (200 ng/10⁶ cells, Kyowa Medex). After staining with FITC-labeled secondary antibody (anti-mouse IgM, 100 ng/10⁶ cells, Pharmingen), the cells were analyzed by flow cytometry.

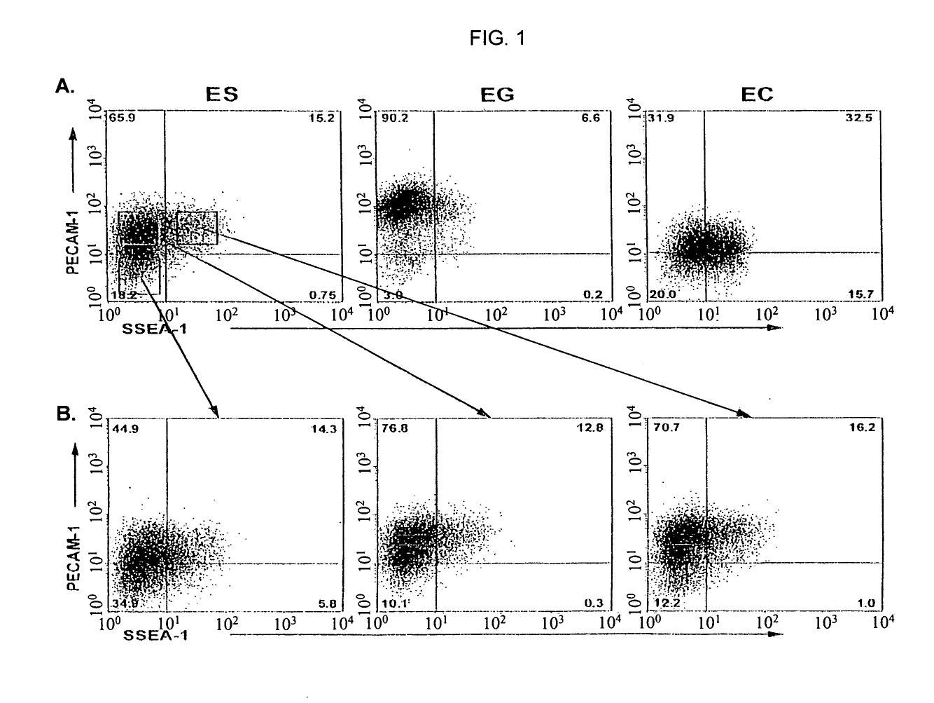

The ES cell staining patterns for various cell-surface markers demonstrated large differences in PECAM-1 and SSEA-1 expression (FIG. 1). This figure shows double-staining patterns after staining with PECAM-1 and SSEA-1, comparing the staining of ES cells, EG cells (TM1), and EC cells (F9). Of the ES cells, about 80% were PECAM-1 positive and among these cells 15.2% were SSEA-1 positive. Notably, almost all of the SSEA-1 positive cells were PECAM-1 positive. The EG cells demonstrated staining patterns similar to those of ES cells. However, their PECAM-1 expression was generally higher, while the percentage of the SSEA-1 positive cells was generally lower. The EC cells had a lower level of PECAM-1 expression than either of the other two cell lines, however demonstrated a higher percentage of SSEA-1 positive cells.

The present inventors then separated the ES cells into three subpopulations. Cells belonging to each subpopulation were cultured separately and then collected after four days. The appearance of cells from different subpopulations was then analyzed. The results showed that in each subpopulation, cells of the other two subpopulations appeared, thus indicating the reversible expression of PECAM-1 and SSEA-1. However, the PECAM-1 negative cells appeared with low frequency and grew much more slowly than the PECAM-1 positive cells. This suggests that PECAM-1 negative cells can be reconstructed from only one portion of a subpopulation.

### Example 2

### The Expression of Differentiation Markers is Increased in PECAM-1 Negative Cells

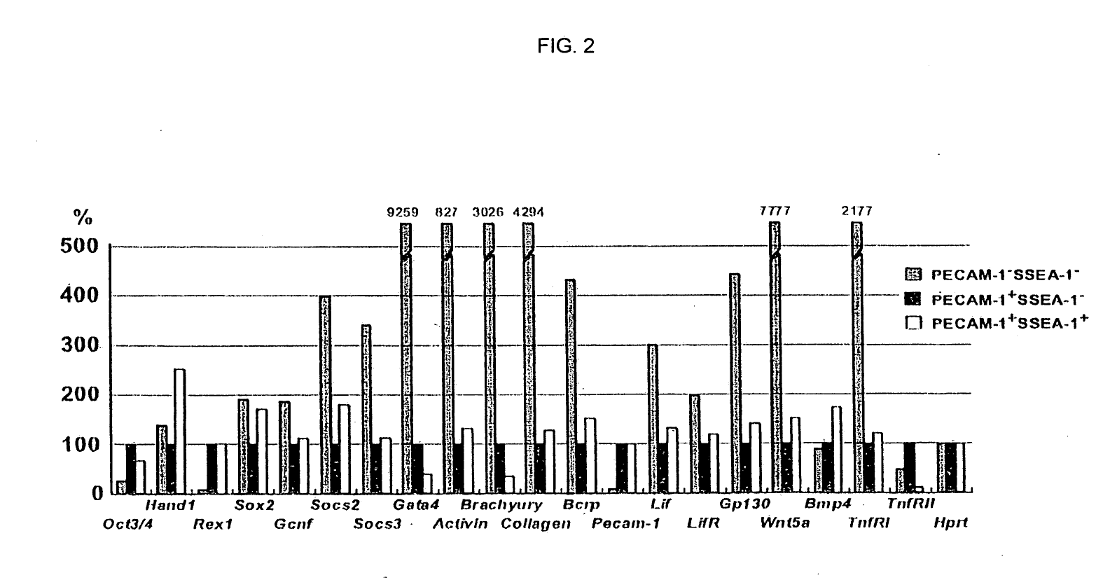

Total RNA was purified from the sorted cells. After DNase treatment, cDNA was synthesized using a standard method. Quantitative PCR was carried out using a LightCycler (Roche). The number of cycles required for the first amplification was used to set a value of 100 for PECAM-1⁺SSEA-1⁻. Using this as a base, relative values corrected using Hprt values were obtained. Thus differences in the expression of genes that regulate and reflect differentiation were analysed for the populations PECAM-1⁻SSEA-1⁻, PECAM-1⁺SSEA-1⁻, and PECAM-1⁺SSEA-1⁺ (FIG. 2).

The results showed that PECAM-1 negative cells had increased expression of differentiation marker genes such as Gata4 (primitive endoderm, heart, smooth muscle), Collagen type IV (primitive endoderm), Activin (epiblast) and Brachyury (mesoderm). On the other hand, gene expression of Oct3/4 and Rex1 was reduced. These results confirm that the cell population are about to differentiate. The PECAM-1⁺SSEA-1⁻ and PECAM-1⁺SSEA-1⁺ cells generally showed similar values. However, there were differences in the gene expression of some markers, including Gata4, Brachyury, Bmp4, and TnfRII.

### Example 3

### Oct3/4 is Not Directly Involved in the Regulation of PECAM-1 Expression

ZHBTc4, a cell line of ES cells, was cultured by standard methods on gelatin-coated dishes and in ES medium with 20% FCS. After addition of Dox (1 mg/ml), cells were collected every 12 hours, up to 48 hours. PECAM-1 expression was then determined by flow cytometry. Total RNA was prepared and quantitative RT-PCR was used to investigate gene expression over time. The level of expression prior to induction was set as 100, and the values corrected with Hprt were indicated. The conditions for flow cytometry and quantitative RT-PCR were the same as for Examples 1 and 2.

The possibility of Oct3/4 involvement in the regulation of PECAM-1 expression was studied using ZHBTc4 ES cells, a line of cells displaying tetracyclin-induced regulation of Oct3/4 expression (Niwa, H., Miyazaki, J. and Smith, A. G.: Nat Genet 24, 372-6 (2000)) (supplied by Dr. Niwa of Riken).

Time-courses of PECAM-1 expression indicated high level PECAM-1 expression prior to induction. Since both Oct3/4 alleles have been destroyed in ZHBTc4 cells, this high level of PECAM-1 expression shows that Oct3/4 is not involved in direct regulation of PECAM-1 expression. A decrease in PECAM-1 expression was confirmed about 36 hours after induction, and the peak had completely shifted after 48 hours. This timing corresponded to changes in cell adhesion characteristics and morphology.

Time-courses of the gene expression of Oct3/4, PECAM-1, Rex1, and Hand1 were also studied. Twelve hours after induction, Oct3/4 expression decreased to 1/200 or less of its original level. Large changes were also observed in Rex1 and Hand1, which are directly regulated by Oct4. However, PECAM-1 showed no change at 12 hours after induction. Differences were observed after 24 hours and beyond. This result suggests that PECAM-1 expression in ES cells is not directly controlled by Oct3/4.

### Example 4

### PECAM-1 Positive Cells are Incorporated into the ICM (Inner Cell Mass)

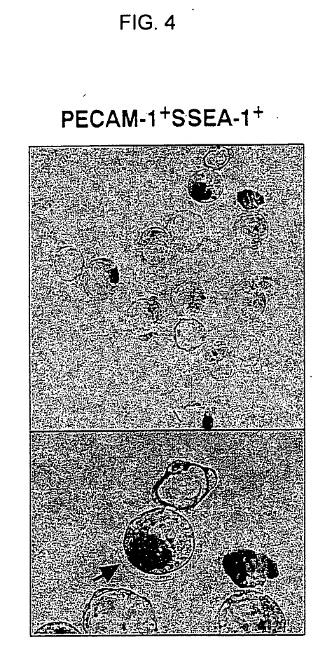

ICR mouse embryos in the eight-cell stage (2.5 d.p.c.) were used as host embryos. After ES cell injection, the embryos were cultured in M16 medium with 10% FCS. After 18 or 36 hours, the embryos were fixed in a solution of 0.2% NP-40 and 0.1% glutaraldehyde-PBS to allow for X-gal staining. Some of these embryos were transferred into the uteri of recipient mice (ICR), harvested four days later (6.0 to 7.0 d.p.c.) and then stained with X-gal. FIG. 4 shows X-gal-stained embryos 36 hours after ES cell injection.

These results showed that a high percentage of PECAM-1 positive cells were incorporated into the ICM chimeric blastocyst (40.5% in PECAM-1⁺SSEA-1⁻ cells and 50% in PECAM-1⁺SSEA-1⁺ cells). On the other hand, PECAM-1 negative cells tended to be localized in the primitive endoderm (25.7%) and the trophectoderm (65.7%) (Table 1).

TABLE 1
Comparison of the degrees of chimera formation in three
cell populations injected into blastocyst

| | Total | Number of | β-gal⁺ tissues | | | |
|---|---|---|---|---|---|---|
| | number of | β-gal⁺ | | | | No |
| Cells injected | embryos | embryos | ICM | PE | TE | incorporation |
| PECAM-1⁻SSEA-1⁻ | 118 | 35 (29.7) | 0 (0) | 9 (25.7) | 23 (65.7) | 3 (8.6) |
| PECAM-1⁺SSEA-1⁻ | 228 | 42 (18.4) | 17 (40.5) | 9 (21.4) | 12 (28.6) | 4 (9.5) |
| PECAM-1⁺SSEA-1⁺ | 190 | 48 (25.3) | 24 (50) | 9 (18.8) | 8 (16.7) | 7 (4.6) |

Abbreviations:
PE, primitive endoderm;
TE, trophectoderm

### Example 5

### PECAM-1⁺SSEA-1⁺ Cells Differentiate to the Epiblast at High Rates

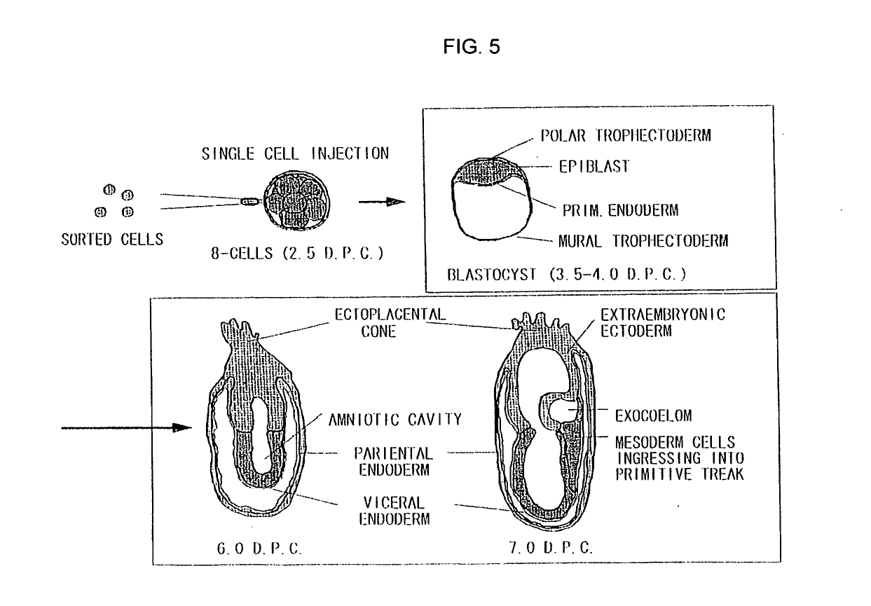

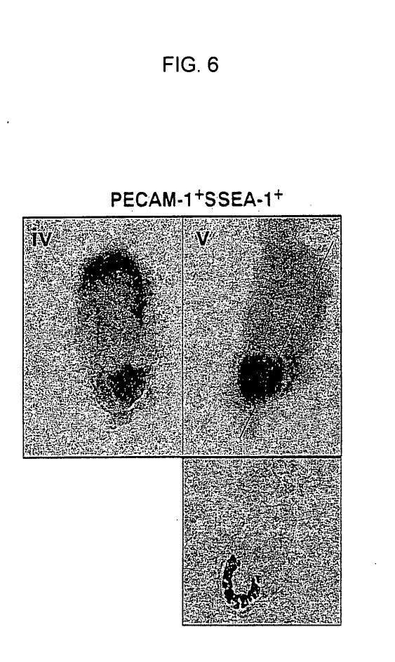

The percentage and localization of the ES cell contribution in 6.0 to 7.0 d. p. c. embryos were studied. The results showed that PECAM-1⁺SSEA-1⁺ cells in all of the embryos (16.7%) in which β-gal positive cells were detected differentiated to, and occupied the majority of, the epiblast (FIG. 6 iv, v). On the contrary, PECAM-1⁻SSEA-1⁻ and PECAM-1⁺SSEA-1⁻ cells produced smaller numbers of β-gal positive embryos, suggesting the possibility of cell loss during the developmental process. When differentiation to the epiblast was observed, the ratio of tissue occupation was low. In addition, differentiation to tissues other than the epiblast, such as to the viceral endoderm and parietal endoderm, was observed. FIG. 5 schematically shows mouse embryo tissues (6.0 and 7.0 d. p. c.). FIG. 6 shows the X-gal staining patterns of embryos harvested four days after transplantation. Table 2 compares the frequency of appearance of β-gal positive cells.

TABLE 2
Comparison of the degrees of chimera formation in three
cell populations in embryos of 6.0 to 7.0 d.p.c.

| | Decidua/ | Embryos/ | β-gal⁺ | β-gal⁺ tissues | |
|---|---|---|---|---|---|
| Cells injected | Transplantation | Decidua | embryos | EP | Others |
| PECAM-1⁻SSEA 1⁻ | 38/63 (60.3) | 33 (86.8) | 3 (9.1) | 1 (3.0) | 2 (6.1) |
| PECAM-1⁺SSEA-1⁻ | 36/61 (59.0) | 28 (77.8) | 1 (3.6) | 1 (3.5) | 0 (0) |
| PECAM-1⁺SSEA-1⁺ | 43/61 (70.5) | 36 (83.7) | 6 (16.7) | 6 (16.7) | 0 (0) |

While this invention has been particularly shown and described with references to preferred embodiments thereof, it will be understood by those skilled in the art that various changes in form and details may be made therein without departing from the scope of the invention encompassed by the appended claims.

1. A method of sorting undifferentiated cells, wherein said method comprises contacting undifferentiated cells with an antibody to a cell-surface antigen, and sorting the undifferentiated cells according to the presence or absence of the binding to the antibody.

2. The method of claim 1, wherein the cell-surface antigen is selected from the group consisting of PECAM-1, SSEA-1, SSEA-3, and SSEA-4.

3. The method of claim 1, wherein the undifferentiated cells are embryonic stem (ES) cells derived from mammals or embryonic germ (EG) cells derived from mammals.

4. The method of claim 1, wherein the undifferentiated cells are transgenic.

5. The method of claim 2, wherein undifferentiated cells that bind to an antibody to PECAM-1 are sorted out.

6. The method of claim 5, wherein undifferentiated cells that bind to both an antibody to PECAM-1 and an antibody to SSEA-1 are sorted out.

7. The method of claim 5, wherein undifferentiated cells that bind to an antibody to PECAM-1, an antibody to SSEA-3, and an antibody to SSEA-4 are sorted out.

8. An undifferentiated cell obtained by the method of claim 5.

9. An isolated undifferentiated cell binding to an antibody to PECAM-1.

10. An isolated undifferentiated cell binding to an antibody to PECAM-1 and an antibody to SSEA-1.

11. An isolated undifferentiated cell binding to an antibody to PECAM-1, an antibody to SSEA-3, and an antibody to SSEA-4.

12. A host embryo comprising the undifferentiated cell of claim 8.

13. A host embryo comprising the undifferentiated cell of claim 8, wherein the embryo is constructed by injection of the undifferentiated cell of claim 8 into a fertilized embryo, or aggregation of that undifferentiated cell with a fertilized embryo.

14. A host embryo comprising the undifferentiated cell of claim 8, wherein said embryo is constructed by injection or fusion of the undifferentiated cell of claim 8 to an enucleated unfertilized oocyte.

15. A differentiated tissue complex derived from the undifferentiated cell of claim 8.

16. A differentiated tissue complex derived from the undifferentiated cell of claim 8, wherein said complex is constructed by inducing differentiation by in vitro culture or in vivo transplantation of the undifferentiated cell of claim 8.

17. A surrogate mother comprising the embryo of claim 12.

18. A fetus, baby, or descendants obtained from the surrogate mother of claim 17.

19. A method of preparing a chimeric embryo comprising the steps of:
a) introducing a ES cell which expresses PECAM-1 and SSEA-1 into a host embryo to be harvested; and
b) selecting a chimeric embryo obtained in step (a).

20. The method of claim 19, wherein the ES cell in step (a) is introduced into a host embryo by injecting a ES cell which expresses PECAM-1 and SSEA-1 into the host embryo.

21. The method of claim 19, wherein the ES cell in step (a) is introduced into a host embryo by aggregating a host embryo which a zona pellucida has been removed with a ES cell which expresses PECAM-1 and SSEA-1.

22. The method of claim 19 wherein the ES cell and host embryo are from mouse.

23. A method of preparing a chimeric mouse comprising the steps of:
a) transferring the embryo prepared by the method of claim 22 into a surrogate mother to be harvested; and
b) selecting a chimeric fetus born of the surrogate mother obtained in step (a).

24. A method of isolating undifferentiated cells which are capable of differentiating into an epiblast of a blastocyst comprising:
a) sorting a population of undifferentiated cells according to the presence or absence of the cell surface expression of PECAM-1; and
b) collecting the undifferentiated cells that express PECAM-1 on their cell surface, wherein said undifferentiated cells so, isolated differentiate into an epiblast of a blastocyst at a higher rate than unsorted undifferentiated cells.

25. The method of claim 24, wherein the undifferentiated cells so isolated are from mouse and further express SSEA-1 on their cell surface.

26. The method of claim 24, wherein the undifferentiated cells are embryonic stem (ES) cells derived from mammals or embryonic germ (EG) cells derived from mammals.

27. The method of claim 24, wherein the undifferentiated cells so isolated are transgenic mouse undifferentiated cells.

28. The method of claim 24, wherein the presence or absence of the cell surface expression of PECAM-1 is detected by binding of an antibody to PECAM-1.

29. The method of claim 25, wherein the expression of SSEA-1 on the mouse undifferentiated cells so isolated is detected by binding of an antibody to SSEA-1.
